(19) United States
(12) Patent Application Publication
(10) Pub. No.: US 2020/0105403 A1
(43) Pub. Date: Apr. 2, 2020

(54) HOSPITAL SUPPORT APPARATUS AND OPERATION METHOD AND OPERATION PROGRAM OF HOSPITAL SUPPORT APPARATUS

(71) Applicant: FUJIFILM Corporation, Tokyo (JP)

(72) Inventors: Hirona YUMBE, Kanagawa (JP); Akemi ODA, Kanagawa (JP); Haruyasu NAKATSUGAWA, Kanagawa (JP)

(73) Assignee: FUJIFILM Corporation, Tokyo (JP)

(21) Appl. No.: 16/579,857

(22) Filed: Sep. 24, 2019

(30) Foreign Application Priority Data
Sep. 27, 2018 (JP) 2018-181293

(51) Int. Cl. G16H 40/20; G16H 40/40; G16H 40/60

(52) U.S. Cl. CPC G16H 40/20; G16H 10/60; G16H 40/60; G16H 40/40

(57) ABSTRACT

The hospital support apparatus performs control for displaying, on a display device, a first table displaying a current stage of a subject among a plurality of stages that the subject goes through in a hospital for each of the subjects. The first table is displayed on the display device side by side with a second table that displays running information indicating a running status of each of a plurality of devices used for the medical treatment or the examination for each device. Correspondence relationship information indicating a correspondence relationship between a target subject of which the current stage indicated in the first table is a stage of the medical treatment or the examination, and the device assigned to the target subject in the second table, is displayed in the first table and the second table.

### CROSS-REFERENCE TO RELATED APPLICATIONS

The present application claims priority under 35 U.S.C. § 119 to Japanese Patent Application No. 2018-181293 filed on Sep. 27, 2018. The above application is hereby expressly incorporated by reference, in its entirety, into the present application.

### BACKGROUND

### 1. Technical Field

The present disclosure relates to a hospital support apparatus and an operation method and an operation program of a hospital support apparatus.

### 2. Description of the Related Art

In a hospital or an animal hospital (hereinafter simply referred to as the hospital), medical treatment or examination is performed on a subject which is a patient or a diseased animal during a hospital visit. Although various devices are used in the medical treatment or the examination, since the number of such devices is limited, naturally, for example, it is difficult to perform the same examination with the same device in parallel on all of the subjects during the hospital visit. Therefore, in a case where the number of subjects is larger than the number of devices, a situation such as waiting for examination occurs.

In systems of JP2017-010165A and JP2010-124937A, in the examination of a subject, a device assigned to each subject is displayed and a device running status screen (a monitoring screen of the dialysis apparatus in JP2017-010165A, and examination status information in JP2010-124937A) displaying the current running status of the device is displayed on a display device. By using such a device running status screen, it is possible to grasp a waiting status and the like of the examination using the device.

### SUMMARY

By the way, in the hospital, there are a plurality of stages that the subject goes through during the hospital visit, such as reception and accounting in addition to the medical treatment and the examination, and the medical treatment and the examination are one of the plurality of stages. Since the medical treatment and the examination are main services performed by the hospital to the subject, displaying the device running status as in JP2017-010165A and JP2010-124937A is useful for grasping a progress status of individual stages of the medical treatment and the examination.

However, it is not possible to grasp the overall progress status regarding all stages that the subject goes through in the hospital only by grasping the progress status of the individual stages of the medical treatment and the examination. For smooth operation of the hospital, it is preferable to be able to grasp not only the individual stages but also the overall progress status.

Therefore, in a case where another system is used to display the overall progress status regarding all the stages that the subject goes through in the hospital in addition to the system for displaying the device running status screen disclosed in JP2017-010165A and JP2010-124937A, it is possible to grasp both an individual progress status and overall progress information.

However, in that case, there is a problem of being extremely complicated because it is necessary to grasp information on both the individual progress status and the overall progress information through respective screens of the plurality of systems. In addition, in the plurality of screens of different systems, the correspondence relationship between the individual progress status and the overall progress information cannot be grasped at a glance, and thus there is also a problem that quick information grasping is hindered.

The present disclosure has been made in view of the above circumstances, and an object of the present disclosure is to provide a hospital support apparatus and an operation method and an operation program of a hospital support apparatus capable of easily and quickly grasping both an overall progress status of services and a progress status of individual medical treatment or examination, which are performed by a hospital to a subject during a hospital visit.

In order to achieve the above object of the present disclosure, there is provided a hospital support apparatus comprising: a first controller that performs control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination; a second controller that performs control for displaying a second table side by side with the first table on the display device, the second table displaying running information indicating a running status of each of a plurality of devices used for the medical treatment or the examination for each device; and a third controller that performs control such that the first table and the second table display correspondence relationship information indicating a correspondence relationship between a target subject of which the current stage indicated in the first table is a stage of the medical treatment or the examination, and the device assigned to the target subject in the second table.

It is preferable that in the first table, the plurality of stages are arranged in a sequence of time, and the first controller moves a display indicating the current stage of each subject according to a progress status of each subject.

It is preferable that the correspondence relationship information includes a first mark visually emphasizing the target subject in the first table, and a second mark visually emphasizing the device assigned to the target subject in the second table.

It is preferable that, regarding the target subject, the first table displays at least one of subject identification information of the target subject or an examination item scheduled to be performed for the target subject, and the first mark is attached to at least one of the subject identification information or the examination item.

It is preferable that the second table displays subject identification information of the subject assigned to each device in addition to device identification information of the device, and the second mark is attached to at least one of the device identification information or the subject identification information.

It is preferable that the first mark and the second mark are displayed in a display mode in which each of the target subjects is identified.

It is preferable that, by changing a display mode of the second mark for each of a plurality of the target subjects assigned to the device, the second table indicates a state of each target subject among a plurality of the states including a first state in which the medical treatment or the examination corresponding to the device is being performed, a second state in which the medical treatment or the examination corresponding to the device is being prepared, and a third state in which the examination is completed, and a display mode of the first mark is also changed in conjunction with the display mode of the second mark.

It is preferable that the plurality of states include a fourth state, and the fourth state is an immediately-after-completion state, after the medical treatment or the examination of the target subject is completed and before a medical treatment or an examination of a next target subject is started in the device.

It is preferable that the first table displays a plurality of examination items in a display section of the stage of the examination.

It is preferable that in the display section, the plurality of states are displayed for each of the plurality of examination items.

It is preferable that the second table displays an error message indicating an error occurring for each device.

It is preferable that the error message includes at least one of an error message indicating an error occurring in the device or an error message indicating an input mistake of an examination target to be set in the device.

In order to achieve the above object of the present disclosure, there is provided an operation program of a hospital support apparatus causing a computer to function as the hospital support apparatus comprising: a first controller that performs control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination; a second controller that performs control for displaying a second table side by side with the first table on the display device, the second table displaying running information indicating a running status of each of a plurality of devices used for the medical treatment or the examination for each device; and a third controller that performs control such that the first table and the second table display correspondence relationship information indicating a correspondence relationship between a target subject of which the current stage indicated in the first table is a stage of the medical treatment or the examination, and the device assigned to the target subject in the second table.

In order to achieve the above object of the present disclosure, there is provided an operation method of a hospital support apparatus, the method comprising: a first control step of performing control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination; a second control step of performing control for displaying a second table side by side with the first table on the display device, the second table displaying running information indicating a running status of each of a plurality of devices used for the medical treatment or the examination for each device; and a third control step of performing control such that the first table and the second table display correspondence relationship information indicating a correspondence relationship between a target subject of which the current stage indicated in the first table is a stage of the medical treatment or the examination, and the device assigned to the target subject in the second table.

According to the present disclosure, it is possible to easily and quickly grasp both an overall progress status of services and a progress status of individual medical treatment or examination, which are performed by a hospital to a subject during a hospital visit.

### BRIEF DESCRIPTION OF THE DRAWINGS

Exemplary embodiments according to the technique of the present disclosure will be described in detail based on the following figures, wherein:

FIG. 1 is a plan view showing an example of a configuration of an animal hospital;

FIG. 2 is a block diagram showing an example of a configuration of a hospital support system;

FIG. 3 is a block diagram showing an example of a hardware configuration of an operation terminal;

FIG. 4 is a block diagram showing an example of a functional configuration of the operation terminal;

FIG. 5 is a block diagram showing an example of a hardware configuration of an examination management terminal;

FIG. 6 is a block diagram showing an example of hardware configurations of a waiting room monitor, a check-up monitor, and a management monitor;

FIG. 7 is a flowchart showing an example of hospital support processing performed by the operation terminal;

FIG. 8 is an explanatory diagram for describing examples of a status management table and an examination device running status table;

FIG. 9 is an explanatory diagram for describing information displayed in the status management table;

FIG. 10 is an explanatory diagram for describing information displayed in the examination device running status table;

FIG. 11 is a diagram showing a state of the status management table and the examination device running status table changed with time from the state of FIG. 8;

FIG. 12 is a block diagram showing another example of the configuration of the hospital support system;

FIG. 13 is a diagram showing Modification Example 1 of a first embodiment;

FIG. 14 is a diagram showing Modification Example 2 of the first embodiment;

FIG. 15 is a diagram showing a second embodiment;

FIG. 16 is a diagram showing a third embodiment;

FIG. 17 is a diagram showing a state changed with time from FIG. 16 in the third embodiment;

FIG. 18 is a diagram showing a fourth embodiment;

FIG. 19 is a diagram showing a modification example of the fourth embodiment;

FIG. 20 is a diagram showing a fifth embodiment;

FIG. 21 is a diagram showing a sixth embodiment; and

FIG. 22 is a diagram showing a modification example of the sixth embodiment.

### DETAILED DESCRIPTION

Hereinafter, an embodiment for implementing a technology of the present disclosure will be described in detail with reference to drawings.

### First Embodiment

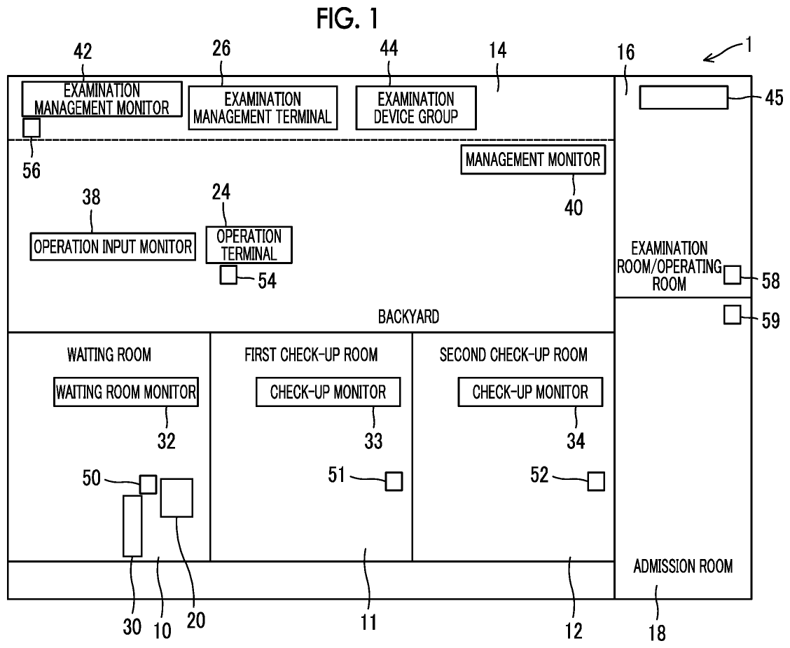

First, an example of an animal hospital which is a support target of a hospital support system of the present embodiment will be described with reference to FIG. 1. FIG. 1 shows a plan view of an example of the animal hospital which is the support target of the hospital support system of the present embodiment.

As shown in FIG. 1, an animal hospital 1 is provided with a waiting room 10, a first check-up room 11, a second check-up room 12, a backyard 14, an examination room/operating room 16 that serves as both an examination room and an operating room, and an admission room 18.

In the waiting room 10, a reception for receiving an animal (hereinafter, referred to as a “diseased animal” regardless of morbidity) which is an example of a subject that visits the animal hospital 1 is provided. In the waiting room 10, a reception/accounting terminal 20 and a reception/accounting terminal monitor 30 to be used for receiving the diseased animal or accounting by a staff of the animal hospital 1 such as a veterinarian or a nurse (hereinafter, simply referred to as the “staff”) are disposed. In addition, in the waiting room 10, a waiting room monitor 32 is disposed for an owner of the diseased animal who waits for a check-up to view and listen. The waiting room monitor 32 displays information of contents the owner is interested in. For example, the information includes various advertisements related to the diseased animal, information useful for health care of the diseased animal (information on diseases, medical check-up, pet food, and the like), or an introduction of the animal hospital 1 (an introduction of pet hotels or an introduction related to trimming in a case where the trimming is performed). In addition, the waiting room monitor 32 may display a photo of each diseased animal. For example, the waiting room monitor displays one or a plurality of favorite photos taken by the owner, and thus, it is possible to smoothly facilitate communication between the owners.

In each of the first check-up room 11 and the second check-up room 12, the veterinarian performs a check-up or treatment (curing) on the diseased animals. A check-up monitor 33 for displaying information on check-up contents or information on the diseased animals such as a so-called medical chart is disposed in the first check-up room 11, and a check-up monitor 34 for the same purpose is disposed in the second check-up room 12. Results of examinations using an examination device group 44 and a radiological examination device 45, for example, a result of a blood examination, a radiographic image, and an ultrasound image are also displayed on the check-up monitor 33 and the check-up monitor 34 of the present embodiment according to a command of the staff.

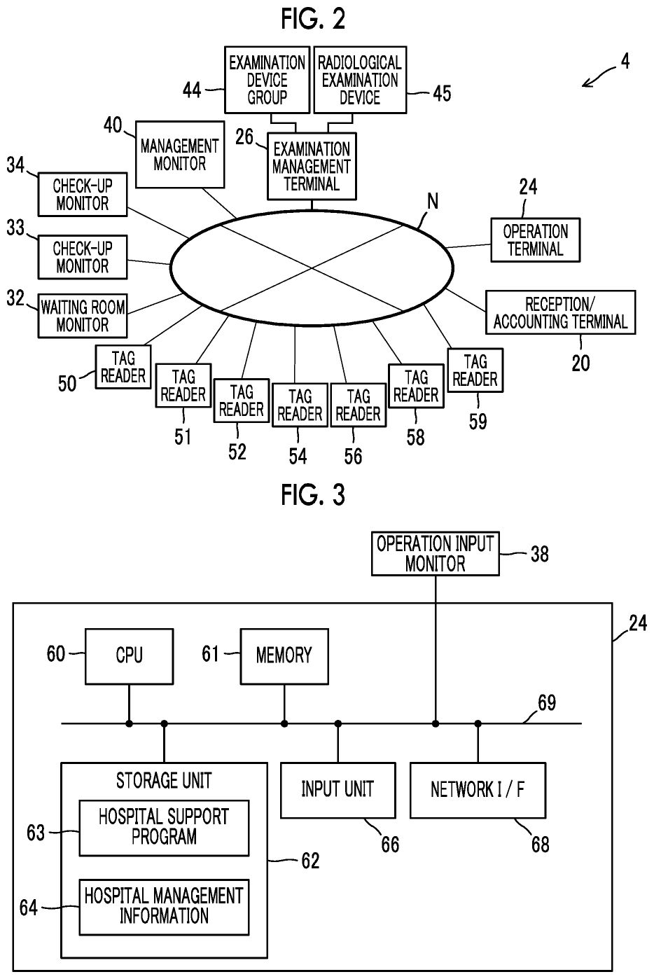

The backyard 14 is provided with an operation terminal 24, an operation input monitor 38, and a management monitor 40, and the staff performs overall management of the animal hospital 1 using the operation terminal 24, the operation input monitor 38, and the management monitor 40. The operation terminal 24 of the present embodiment controls an entire hospital support system 4 (see FIG. 2) used to manage the animal hospital 1. The operation terminal 24 of the present embodiment is an example of a hospital support apparatus according to the embodiment of the present disclosure. The operation input monitor 38 and the management monitor 40 of the present embodiment are examples of a display device according to the embodiment of the present disclosure.

In the backyard 14, the examination or treatment on the diseased animal or a specimen obtained from the diseased animal is performed by the veterinarian or the like. Therefore, the examination device group 44 used for the examination, and an examination management terminal 26 and an examination management monitor 42 for managing the general examination are disposed in the backyard 14. The examination device group 44 includes, for example, a blood chemical examination device, an immunological examination device, a radiological examination device, and an ultrasound examination device. The examination device group is not particularly limited, and may include an endoscopic examination device. As a specific example of each of the blood chemical examination device and the immunological examination device, there is a product name “DRI-CHEM (registered trademark)” manufactured by FUJIFILM Corporation.

In the examination room/operating room 16, the veterinarian performs imaging of a radiographic image of the diseased animal or surgery on the diseased animal. The radiological examination device 45 for imaging the radiographic image of the diseased animal is disposed in the examination room/operating room 16, and the radiological examination device 45 is managed by using the examination management terminal 26 provided in the backyard 14.

A plurality of diseased animals can be admitted (accommodated) in the admission room 18, and a plurality of animals can stay in the admission room as a so-called pet hotel.

As shown in FIG. 1, a tag reader 50, a tag reader 51, a tag reader 52, tag readers 54 and 56, a tag reader 58, and a tag reader 59 as readers for reading integrated circuit (IC) tags storing identification numbers (to be described in detail) for managing the diseased animals are respectively provided in the waiting room 10, the first check-up room 11, the second check-up room 12, the backyard 14, the examination room/operating room 16, and the admission room 18. In the present embodiment, as an example, an IC tag is included in a check-up card of the diseased animal.

Next, a configuration of the hospital support system 4 of the present embodiment will be described with reference to FIG. 2. As shown in FIG. 2, in the hospital support system 4 of the present embodiment, the reception/accounting terminal 20, the operation terminal 24, the examination management terminal 26, the waiting room monitor 32, the check-up monitor 33, the check-up monitor 34, the management monitor 40, and the tag readers 50, 51, 52, 54, 56, 58, and 59 which are described above are communicably connected to a so-called intra-hospital network N provided in the animal hospital 1. Further, the examination management terminal 26 and each of the examination device group 44 and the radiological examination device 45 are communicably connected without the network N. The communication between the examination management terminal 26 and each of the examination device group 44 and the radiological examination device 45 may be performed in a wireless communication manner or in a wired communication manner.

Next, a hardware configuration of the operation terminal 24 of the present embodiment will be described with reference to FIG. 3. As shown in FIG. 3, the operation terminal 24 includes a central processing unit (CPU) 60, a memory 61 as a temporary storage area, and a non-volatile storage unit 62. The operation terminal 24 also includes an input unit 66 such as a keyboard or a mouse, and a network interface (I/F) 68 connected to the network N. The CPU 60, the memory 61, the storage unit 62, the input unit 66, the network I/F 68, and the operation input monitor 38 are communicably connected to a bus 69.

The memory 61 is realized by a random access memory (RAM) or the like. The memory 61 is a work memory for work used in a case where the CPU 60 executes various processes.

The storage unit 62 is realized by a hard disk drive (HDD), a solid state drive (SSD), a flash memory, and the like. A hospital support program 63 is stored in the storage unit 62 as a storage medium. The CPU 60 reads out the hospital support program 63 from the storage unit 62 and then develops the readout program into the memory 61 and executes the developed hospital support program 63. The hospital support program 63 is an example of an operation program of a hospital support apparatus according to the embodiment of the present disclosure. Further, hospital management information 64 including diseased animal information on the diseased animal which is recorded in a so-called medical chart or various information for managing the entire animal hospital 1 is stored in the storage unit 62. The hospital management information 64 includes, for example, appointment information for medical treatment or examination of the diseased animal.

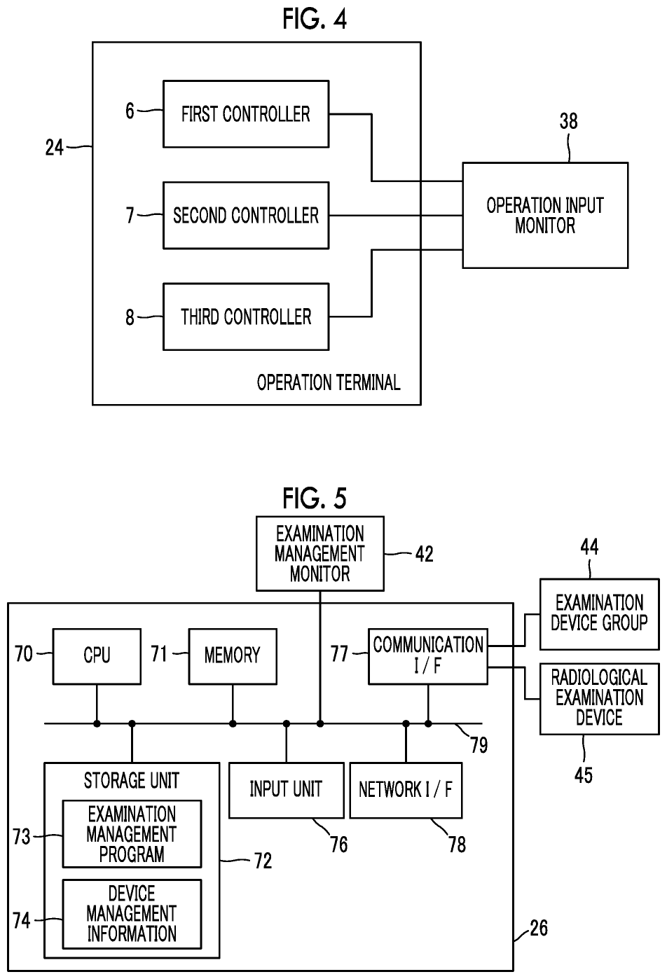

A functional configuration of the operation terminal 24 of the present embodiment will be described with reference to FIG. 4. As shown in FIG. 4, the operation terminal 24 includes a first controller 6, a second controller 7, and a third controller 8. The CPU 60 executes the hospital support program 63, and thus, the operation terminal functions as the first controller 6, the second controller 7, and the third controller 8.

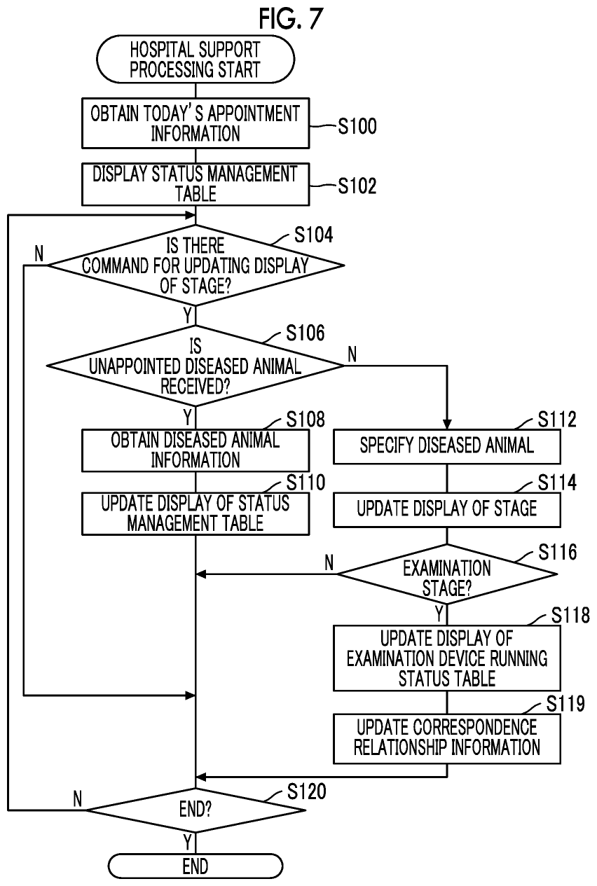

The first controller 6 performs control for displaying, on the operation input monitor 38 and the management monitor 40, a status management table 100 (see FIG. 7) displaying a current stage of the diseased animal among a plurality of stages that the diseased animal goes through in the hospital from arrival of the diseased animal at the animal hospital 1 to completion of accounting for each of the diseased animals. The plurality of stages include the stage of the medical treatment or the examination. The status management table 100 is an example of a first table.

The second controller 7 performs control for further displaying, on the operation input monitor 38 and the management monitor 40 side by side with the status management table 100, an examination device running status table 102 displaying running information indicating a running status of each of a plurality of devices used for the medical treatment or the examination for each device. The examination device group 44 and the radiological examination device 45 are examples of a plurality of devices used for the examination. The examination device running status table 102 is an example of a second table.

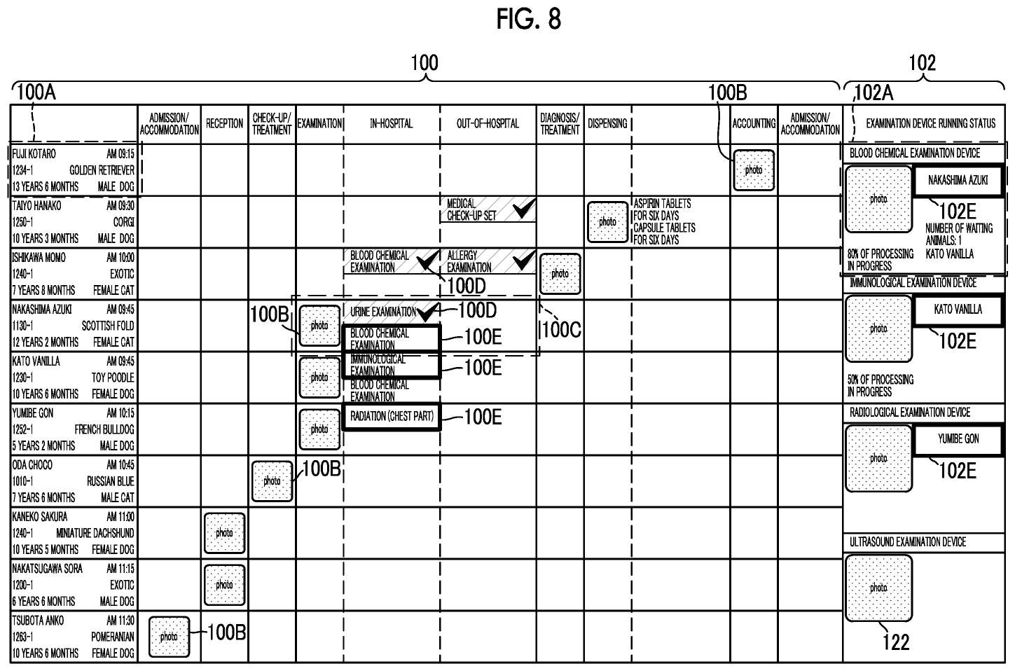

The third controller 8 performs control such that the status management table 100 and the examination device running status table 102 display correspondence relationship information (see reference signs 100E and 102E in FIG. 8 and the like) indicating a correspondence relationship between a target diseased animal (an example of a target subject) of which the current stage indicated in the status management table 100 is the stage of the medical treatment or the examination, and the device assigned to the target diseased animal in the examination device running status table 102.

A hardware configuration of the examination management terminal 26 of the present embodiment will be described with reference to FIG. 5. As shown in FIG. 5, the examination management terminal 26 includes a CPU 70, a memory 71 as a temporary storage area, and a non-volatile storage unit 72. In addition, the examination management terminal 26 includes an input unit 76 such as a keyboard or a mouse, a communication I/F 77 for communicating with the examination device group 44 and the radiological examination device 45, and a network I/F 78 connected to the network N. The CPU 70, the memory 71, the storage unit 72, the input unit 76, the communication I/F 77, the network OF 78, and the examination management monitor 42 are communicably connected to a bus 79.

The memory 71 is realized by a RAM or the like. The memory 71 is a work memory for work which is used in a case where the CPU 70 executes various processes. The storage unit 72 is realized by an HDD, an SSD, a flash memory, and the like. An examination management program 73 is stored in the storage unit 72 as the storage medium. The CPU 70 reads out an examination management program 73 from the storage unit 72, develops the readout program into the memory 71, and executes the developed examination management program 73. The storage unit 72 also stores device management information 74 for managing the examination device group 44 and the radiological examination device 45.

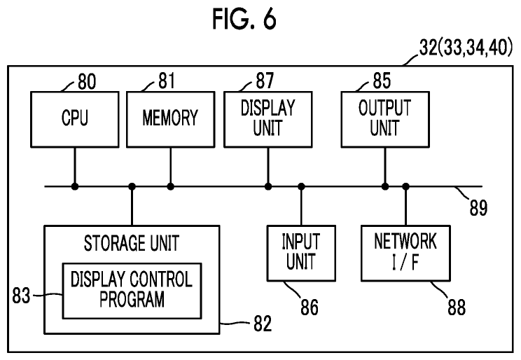

Furthermore, a hardware configuration of the waiting room monitor 32 of the present embodiment will be described with reference to FIG. 6. As shown in FIG. 6, the waiting room monitor 32 includes a CPU 80, a memory 81 as a temporary storage area, and a non-volatile storage unit 82. The waiting room monitor 32 includes an output unit 85 such as a speaker that outputs sound, an input unit 86 such as a remote controller, a display unit 87 such as a liquid crystal display, and a network I/F 88 connected to the network N. The CPU 80, the memory 81, the storage unit 82, the output unit 85, the input unit 86, the display unit 87, and the network I/F 88 are communicably connected to a bus 89.

The memory 81 is realized by a RAM or the like. The memory 81 is a work memory for work which is used in a case where the CPU 80 executes various processes. The storage unit 82 is realized by an HDD, an SSD, a flash memory, and the like. A display control program 83 is stored in the storage unit 82 as the storage medium. The CPU 80 reads out the display control program 83 from the storage unit 82, develops the readout program into the memory 81, and executes the developed display control program 83. The CPU 80 performs control for displaying various information on the display unit 87 according to the control of the operation terminal 24 by executing the display control program 83.

The hardware configurations of the check-up monitor 33, the check-up monitor 34, and the management monitor 40 are the same as that of the waiting room monitor 32, and thus, the description thereof will be omitted.

Next, the actions and effects of the hospital support system 4 of the present embodiment will be described. In the hospital support system 4 of the present embodiment, the operation terminal 24 controls the entire hospital support system 4 as described above. In the present embodiment, the CPU 60 of the operation terminal 24 executes the hospital support program 63, and thus, hospital support processing shown in FIG. 7 is performed. The hospital support processing shown in FIG. 7 is started, for example, at a timing when a power supply (not shown) is input to the operation terminal 24.

In step S100 of FIG. 7, the CPU 60 of the operation terminal 24 obtains today's appointment information. Specifically, the CPU 60 obtains the diseased animal information on the diseased animals whose visits are appointed today while referring to the hospital management information 64.

In the next step S102, the CPU 60 generates the status management table 100 in which the diseased animals whose visits are appointed today are arranged in a sequence of time in ascending order of appointment time, and displays the generated table on the operation input monitor 38 and the management monitor 40. In the hospital support system 4 of the present embodiment, the CPU 60 of the operation terminal 24 executes the hospital support processing, and thus, the status management table 100 shown in FIG. 8 as an example is displayed on the operation input monitor 38 and the management monitor 40.

As shown in FIG. 8, the status management table 100 is a table in which the plurality of stages that the diseased animals go through in the animal hospital 1 are arranged in a sequence of time. In the status management table 100, the time axis is assigned to a lateral axis, and a plurality of stages are arranged along the lateral axis. In the time axis, the left side shows the past, and the right side shows the future. Of the diseased animal information on the diseased animals, a basic information display section 100A in which basic information such as a name of the diseased animal is displayed is assigned to a vertical axis of the status management table 100, and the basic information display section 100A is arranged along the vertical axis.

Among the plurality of stages, the current stage of the diseased animal is displayed in the status management table 100 for each diseased animal. In the present embodiment, as an example of the plurality of stages, “admission/accommodation”, “reception”, “check-up/treatment”, “examination”, “diagnosis/treatment”, “dispensing”, “accounting”, and “admission/accommodation” are shown in a sequence of time. In principle, all the diseased animals that visit the animal hospital 1 go through these stages in order in the animal hospital 1.

Among these stages, the stage of “admission/accommodation” is an example of a stage related to a visit schedule, a stage related to accommodation, a stage related to admission, and a stage related to discharge from a hospital in the present disclosure. In addition, the stages of “reception”, “check-up/treatment”, “diagnosis/treatment”, and “dispensing” are examples of stages related to medical treatment in the present disclosure, and “examination” is an example of a stage related to the examination of the subject in the present disclosure.

In the status management table 100, an icon 100B representing each diseased animal is displayed at a position of the current stage of each diseased animal among the plurality of stages arranged in the sequence of time. The icon 100B is an example of a display indicating the current stage of each diseased animal. For example, a photo of the diseased animal is used as the icon 100B. In the status management table 100, the first controller 6 moves the icon 100B according to the progress status of each diseased animal. Accordingly, the icon 100B of the diseased animal moves to the right as the stage of the diseased animal progresses.

The photo of the diseased animal, which is used as the icon 100B in the present example, is an example of subject identification information for identifying the diseased animal that is the subject. In a case of using such a photo, for example, there are the following advantages. In the case of using the photo of the diseased animal, data of the photo is provided electronically from the owner of the diseased animal by e-mail or the like. Therefore, at that time, the animal hospital 1 can obtain contact information such as an owner's e-mail address. Such contact information can be used for contacting the owner from the animal hospital 1 about an examination result of the diseased animal, or the like.

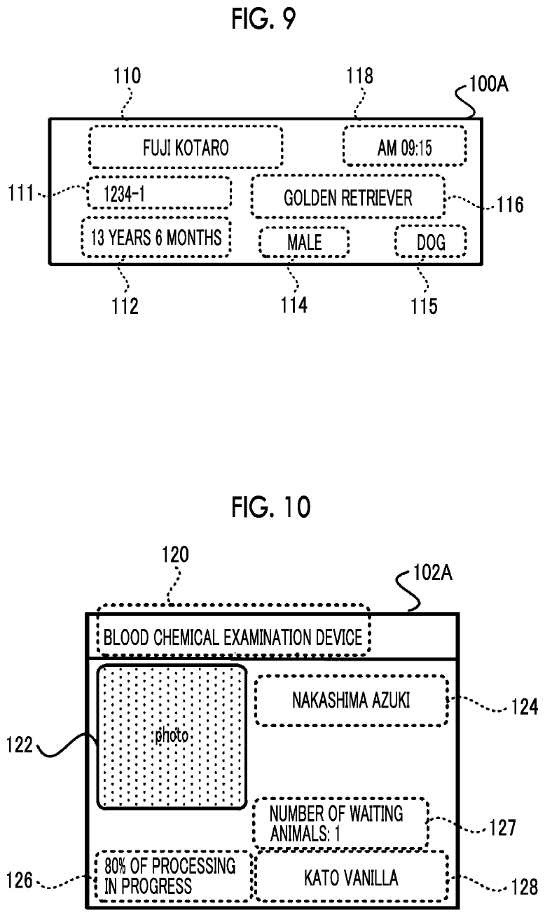

In the present embodiment, examples of the basic information display section 100A include a name 110 of the diseased animal, an identification number 111, an age 112, a gender 114, a type 115, a breed 116, and an appointment time 118, as shown in FIG. 9. The identification number 111 is subject identification information given by the animal hospital 1 to the received diseased animal. The appointment time 118 is changed to the reception time in a case where the diseased animal's reception is completed and the current stage becomes “reception”.

In the next step S104, the CPU 60 determines whether or not a command for updating the display of the current stage is received. In the present embodiment, in a case where identification information is read from the IC tag, each of the tag readers 50, 51, 52, 54, 56, 58, and 59 transmits the identification information to the operation terminal 24, and transmits a command for updating the display of the current stage of the diseased animal at the current point of time. In a case where the command for updating the display of the current stage is received, the CPU 60 updates the display of the current stage of the diseased animal, which corresponds to the received identification information, to the display of the stage corresponding to any of the tag readers 50, 51, 52, 54, 56, 58, and 59 as transmission sources.

In a case where the command for updating the display of the current stage is not received from any of the tag readers 50, 51, 52, 54, 56, 58, and 59 through the network I/F 68, the determination in step S104 is a negative determination, and the CPU proceeds to step S120. Meanwhile, in a case where the command for updating the display of the current stage is received from any one of the tag readers 50, 51, 52, 54, 56, 58, and 59 through the network I/F 68, the determination in step S104 is a positive determination, and the CPU proceeds to step S106.

In step S106, the CPU 60 determines whether or not an unappointed diseased animal is received. Specifically, in a case where an identification number different from an identification number of the diseased animal included in the today's appointment information obtained in step S100 is received from the tag reader 50, the CPU 60 determines that the unappointed diseased animal is received. In a case where the unappointed diseased animal is received, the determination in step S106 is a positive determination, and the CPU proceeds to step S108.

In step S108, the CPU 60 obtains diseased animal information corresponding to the information 100A, which corresponds to the identification information received from the tag reader 50, from the hospital management information 64 stored in the storage unit 62 of the operation terminal 24. In a case where a diseased animal newly visits the animal hospital 1, the staff stores diseased animal information on the diseased animal that visits the animal hospital as the hospital management information 64 in the storage unit 62 by using the reception/accounting terminal 20 or the operation terminal 24.

In the next step S110, the CPU 60 updates the status management table 100 by adding the basic information display section 100A of the unappointed diseased animal, and then proceeds to step S120.

Meanwhile, in step S106, in a case where the unappointed diseased animal is not received, that is, in a case where it is an appointed diseased animal, the determination in step S106 is a negative determination, and the CPU proceeds to step S112.

In step S112, the CPU 60 specifies the diseased animal based on an identification signal received from any of the tag readers 50, 51, 52, 54, 56, 58, and 59.

In the next step S114, the CPU 60 updates the display of the current stage of the diseased animal specified in step S112 on the status management table 100 to the stage corresponding to the transmission source of the identification signal of any of the tag readers 50, 51, 52, 54, 56, 58, and 59.

Specifically, in a case where the diseased animal (and the owner) having the check-up card visits the animal hospital 1, reception processing is first performed by the staff using the reception/accounting terminal 20. In a case where the reception is performed, the IC tag included in the check-up card is read by the tag reader 50 disposed in the waiting room 10, and an identification signal is transmitted from the tag reader 50. Thus, in a case where the identification signal is received from the tag reader 50, the CPU 60 displays the icon 100B of the diseased animal in a position corresponding to “reception” of the status management table 100 in order to display the current stage of the diseased animal as the stage of “reception”.

In a case where the medical treatment of the diseased animal being admitted or accommodated in the admission room 18 of the animal hospital 1 from the previous day is appointed, the icon 100B of the diseased animal is displayed in the position corresponding to “admission/accommodation” as the previous stage of “reception”. Thereafter, in a case where the reception processing is performed by the owner, the icon 100B moves to the stage of “reception” as described above.

In addition, in a case where the reception is completed and the check-up is performed by the veterinarian in the first check-up room 11, the IC tag included in the check-up card is read by the tag reader 51 disposed in the first check-up room 11, and an identification signal from the tag reader 51 is transmitted. In a case where the check-up is performed by the veterinarian in the second check-up room 12, the IC tag included in the check-up card is similarly read by the tag reader 52 disposed in the second check-up room 12, and an identification signal from the tag reader 52 is transmitted. Thus, in a case where the identification signal is received from the tag reader 51 or the tag reader 52, the CPU 60 moves the icon 100B of the diseased animal in the position corresponding to the “check-up/treatment” which is the subsequent stage of “reception” in the status management table 100 in order to display the current stage of the diseased animal as the stage of “check-up/treatment”. The CPU 60 may determine whether or not the identification signal is received from any of the tag reader 51 and the tag reader 52, and may display information indicating that the check-up is performed in any of the first check-up room 11 and the second check-up room 12 in the status management table 100.

In a case where the examination of the diseased animal or the specimen obtained from the diseased animal (hereinafter, referred to as the “examination on the diseased animal”) is performed after the check-up, the IC tag included in the check-up card is read by the tag reader 56 disposed in backyard 14 or the tag reader 58 disposed in the examination room/operating room 16, and an identification signal is transmitted from the tag reader 56 or the tag reader 58. Thus, in a case where the identification signal is received from the tag reader 56 or the tag reader 58, the CPU 60 displays the icon 100B of the diseased animal in the position corresponding to the “examination” which is the subsequent stage of “check-up/treatment” in the status management table 100 in order to display the current stage of the diseased animal as the stage of “examination”.

In addition, in the present embodiment, in a case where the examination on the diseased animal is performed, the management of the entire examination is performed by the examination management terminal 26 as described above. As in the example of the status management table 100 shown in FIG. 8, the CPU 60 obtains an examination item scheduled to be performed for each diseased animal from the examination management terminal 26 and displays the obtained examination item.

In the examination stage display section 100C of the status management table 100, a plurality of examination items scheduled to be performed, such as a “urine examination” and a “blood chemical examination”, are displayed. In the present example, the plurality of examination items are vertically arranged in the examination stage display section 100C.

In addition, the display of the examination stage display section 100C is updated according to the running status of the examination device assigned to each diseased animal for each examination item. The update is performed in conjunction with the update of the examination device running status table 102 as described later. In the example of FIG. 8, the examination items for which the examination is completed are displayed, for example, grayed out as indicated by hatching, and a check mark 100D is attached beside the examination items. Accordingly, the examination item in a state in which the examination is completed and the examination item in a state in which the examination is being performed or not yet examined can be identified. In addition, for the examination item in a state in which the examination is being performed, a thick frame 100E is attached to the examination item. Thereby, the examination item which is being performed is visually emphasized.

In the example shown in FIG. 8, the examination stage display section 100C is divided into a display section representing the contents of the examination (indicated as “in-hospital” in FIG. 8) performed in the animal hospital 1, and a display section representing the contents of the examination (indicated as “out-of-hospital” in FIG. 8) performed outside the animal hospital 1. The examination outside of the animal hospital 1 is, of course, not performed in the animal hospital 1 and is performed outside after an accounting stage of the animal hospital 1. Therefore, regarding the examination outside of the animal hospital 1, it is considered that the examination stage in the animal hospital 1 is completed by having requested examination to an external examination organization from the animal hospital 1.

In a case where the diagnosis or treatment is performed by the veterinarian in the first check-up room 11 after the examination on the diseased animal, the IC tag included in the check-up card is read by the tag reader 51 disposed in the first check-up room 11, and the identification signal is transmitted from the tag reader 51. In a case where the diagnosis or treatment is performed by the veterinarian in the second check-up room 12, the IC tag included in the check-up card is similarly read by the tag reader 52 disposed in the second check-up room 12, and the identification signal is transmitted from the tag reader 52. Thus, in a case where the identification signal is received from the tag reader 51 or the tag reader 52, the CPU 60 moves the icon 100B of the diseased animal in the position corresponding to the “diagnosis/treatment” of the subsequent stage of “examination” in the status management table 100 in order to display the current stage of the diseased animal as the stage of “diagnosis/treatment”. The CPU 60 may determine whether or not the identification signal is received from any of the tag reader 51 or the tag reader 52, and may display information indicating which of the first check-up room 11 and the second check-up room 12 in which the diagnosis or treatment is performed is in the status management table 100.

In a case where the diagnosis or treatment is completed and dispensing is performed, the IC tag included in the check-up card is read by the tag reader 54 disposed in the backyard 14, and an identification signal is transmitted from the tag reader 54. Thus, in a case where the identification signal is received from the tag reader 54, the CPU 60 moves the icon 100B of the diseased animal in the position corresponding to “dispensing” which is the subsequent stage of “diagnosis/treatment” in the status management table 100 in order to display the current stage of the diseased animal as the stage of “dispensing”. In the present embodiment, in a case where medicine to be given to the diseased animal is dispensed, the management of dispensed contents is performed by the operation terminal 24. It is preferable that the CPU 60 obtains information indicating the dispensed contents from the operation terminal 24 and displays the obtained information as in the example of the status management table 100 shown in FIG. 8. The example of the status management table 100 shown in FIG. 8 illustrates a state in which the information indicating the dispensed contents are displayed in line with the stage of “dispensing”.

In a state in which the dispensed medicine is transferred to the owner of the diseased animal, accounting processing related to the medical treatment is performed by the staff by using the reception/accounting terminal 20. In a case where the accounting is performed, the IC tag included in the check-up card is read by the tag reader 50 disposed in the waiting room 10, and an identification signal is transmitted from the tag reader 50. Thus, in a case where the identification signal is received from the tag reader 50, the CPU 60 displays the icon 100B of the diseased animal in the position corresponding to “accounting” which is the subsequent stage of “dispensing” in the status management table 100 in order to display the current stage of the diseased animal as the stage of “accounting”.

Thereafter, in a case where the diseased animal is admitted, the diseased animal together with the check-up card moves to the admission room 18, the IC tag included in the check-up card is read by the tag reader 59 disposed in the admission room 18, and an identification signal is transmitted from the tag reader 59. Thus, in a case where the identification signal is received from the tag reader 59, the CPU 60 displays the icon 100B of the diseased animal in the position corresponding to “admission/accommodation” which is the subsequent stage of “accounting” in the status management table 100 in order to display the current stage of the diseased animal as the stage of “admission/accommodation”.

As described above, in a case where the display of the current stage of diseased animal is updated, in the next step S116, the CPU 60 determines whether the current stage is the stage of “examination”. In a case where the current stage is the stage other than the stage of “examination”, the determination in step S116 is a negative determination, and the CPU proceeds to step S120. Meanwhile, in a case where the current stage is the stage of “examination”, the “determination” in step S116 is a positive determination, and the CPU proceeds to step S118.

In step S118, the CPU 60 updates the examination device running status table 102 displayed on the operation input monitor 38 and the management monitor 40 in order to display the running status of the examination device at the current point of time.

As an example shown in FIG. 8, in the hospital support system 4 of the present embodiment, the examination device running status table 102 together with the status management table 100 are also displayed on the operation input monitor 38 and the management monitor 40. The examination device running status table 102 is a table in which information indicating the running statuses of the examination device group 44 and the radiological examination device 45 managed by the examination management terminal 26 is displayed as a display list of pictures.

The examination device running status table 102 displays running information indicating the running status of each examination device included in the examination device group 44 and each of a plurality of devices including the radiological examination device 45 for each device. The examination device running status table 102 is displayed side by side with the status management table 100. The examination device running status table 102 is an example of the second table.

As shown in FIG. 10, in the present embodiment, the running information is displayed in a running information display section 102A. The running information displayed in the running information display section 102A includes, as an example, a name 120 of the examination device, a photo 122 of the examination device, and a name 124 of the diseased animal being examined by the examination device, a running progress status 126 for examination, the number of diseased animals 127 waiting for examination, and a name 128 of the diseased animal waiting for the examination.

In addition, in the example shown in FIG. 10, the progress degree is shown as a percentage, such as what % of the entire processing as the progress status 126 regarding one examination performed by the examination device. The progress status 126 may be displayed in a form other than a percentage, and for example, a remaining time until the running of the examination device is completed or a scheduled timing when the running of the device is scheduled to be completed may be displayed.

The CPU 60 obtains information from the examination management terminal 26, and controls the display contents of the examination device running status table 102. Specifically, the CPU 60 updates the progress status 126 by obtaining information that is the basis of the running information of the examination device group 44 or the radiological examination device 45 from the examination management terminal 26.

Then, in step S119, the CPU 60 updates the correspondence relationship information indicating the correspondence relationship between the target diseased animal (corresponding to the target subject) of which the current stage indicated in the status management table 100 is the examination stage and the examination device assigned to the target diseased animal in the examination device running status table 102. In the example shown in FIG. 8, the target diseased animals are the diseased animals such as “Nakashima Azuki”, “Kato Vanilla”, and “Yumibe Gon” of which the current stage is the stage of examination. Further, in the example shown in FIG. 8, the devices assigned to the target diseased animals are a blood chemical examination device for “Nakashima Azuki”, and a blood chemical examination device and an immunological examination device for “Kato Vanilla”. For “Yumibe Gon”, a radiological examination device is assigned.

The CPU 60 performs control such that the status management table 100 and the examination device running status table 102 display correspondence relationship information between the target diseased animal and the examination device assigned to the target diseased animal. In the example shown in FIG. 8, the correspondence relationship information is a thick frame 100E visually emphasizing the target diseased animal in the status management table 100 and a thick frame 102E visually emphasizing the device assigned to the target diseased animal in the examination device running status table 102.

The thick frame 100E is an example of the first mark, and the thick frame 102E is an example of the second mark. In this example, the thick frame 100E is attached to the examination item scheduled to be performed for the target diseased animal. Meanwhile, the thick frame 102E is attached to a name, which is an example of subject identification information of the target diseased animal, such as “Nakashima Azuki” or “Kato Vanilla”.

The thick frame 100E and the thick frame 102E are updated in conjunction with each other according to the running status of the examination device. For example, in a case where the examination of one diseased animal is completed in the examination device and the examination of the next diseased animal is started, the target diseased animal indicated by the thick frame 102E is changed in the examination device running status table 102. In conjunction with the change, also in the status management table 100, the target diseased animal indicated by the thick frame 100E is changed.

For example, it is assumed that the examination of “Nakashima Azuki” is completed and the examination of the next “Kato Vanilla” is started in the running information display section 102A of the blood chemical examination device in the examination device running status table 102.

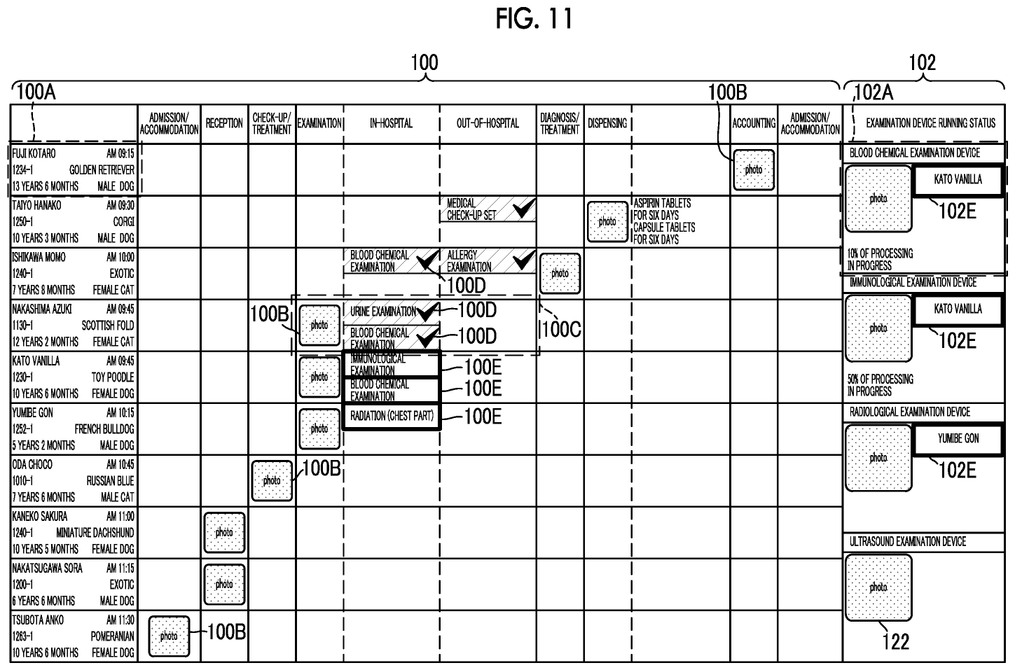

In this case, as shown in FIG. 11, the display of “Nakashima Azuki” disappears from the running information display section 102A, and “Kato Vanilla” displayed as the waiting diseased animal is displayed as a diseased animal in the examination instead of “Nakashima Azuki”. Then, the thick frame 102E is attached to “Kato Vanilla”. In the running information display section 102A, the display of the progress status 126, and the number of diseased animals 127 on waiting (see FIG. 10) are also updated.

In conjunction with the update of the running status, the display of the thick frame 100E of the status management table 100 is also updated in the examination device running status table 102. In a case where the blood chemical examination of “Nakashima Azuki” is completed and the blood chemical examination of “Kato Vanilla” is started, in the examination stage display section 100C of “Nakashima Azuki”, a check mark 100D indicating the completion of the examination is attached to the examination item of the blood chemical examination, and the examination item itself is also hatched. Meanwhile, in the examination stage display section 100C of “Kato Vanilla” where the blood chemical examination is started, the thick frame 100E is displayed in both examination items, because the blood chemical examination is added as an examination item in examination in addition to the immunological examination.

In the running information display section 102A of the examination device running status table 102, the thick frame 102E is attached to the name of the target diseased animal of which the examination is being performed, and the name is visually emphasized. In addition, in an examination stage display section 102C of the status management table 100, the thick frame 100E is attached to the examination item scheduled to be performed for the target diseased animal, so that the target diseased animal of which the examination is being performed is visually emphasized. The correspondence relationship between the name of the target diseased animal and the examination item scheduled to be performed is indicated by the thick frame 100E and the thick frame 102E. It is possible to grasp which examination is being performed for which target diseased animal.

Furthermore, both the thick frame 100E and the thick frame 102E are thick frames having the same form, and the correspondence relationship between the name of the target diseased animal and the examination item scheduled to be performed can be more clearly checked by identity in the form.

In a case where a negative determination is made in step S104 or S116, the CPU 60 proceeds to step S120. In a case where the process in step S119 is ended, the CPU 60 proceeds to step S120. In step S120, the CPU 60 determines whether or not the present hospital support processing is ended. For example, until a switch (not shown) of the operation terminal 24 is turned off, the determination in step S120 is a negative determination, and the CPU returns to step S104 and repeats the processing in steps S106 to S119. Meanwhile, for example, in a case where the switch of the operation terminal 24 is turned off, the determination in step S120 is a positive determination, and the present hospital support processing is ended.

As described above, the operation terminal 24 comprises the first controller 6, the second controller 7, and the third controller 8. The first controller 6 performs control for displaying, on the operation input monitor 38 and the management monitor 40, the status management table 100 displaying the current stage of the diseased animal among the plurality of stages that the diseased animal goes through in the animal hospital 1 from arrival of the diseased animal to completion of accounting for each of the diseased animals. The examination is included in the plurality of stages.

The second controller 7 performs control for displaying, on the operation input monitor 38 and the management monitor 40 side by side with the status management table 100, the examination device running status table 102 displaying the running information indicating the running status of each of the plurality of devices used for the examination for each device.

The third controller 8 performs control such that the status management table 100 and the examination device running status table 102 display correspondence relationship information indicating a correspondence relationship between the target diseased animal of which the current stage indicated in the status management table 100 is the stage of the examination, and the examination device assigned to the target diseased animal in the examination device running status table 102.

Therefore, in the status management table 100, the staff can grasp the overall progress status of the diseased animals in the animal hospital 1. The status management table 100 and the examination device running status table 102 are displayed side by side. Furthermore, correspondence relationship information between the running information of the examination device running status table 102 and the target diseased animal of the status management table 100 is displayed in each of the tables 100 and 102. Accordingly, the staff can grasp the individual progress status in the stage of examination in addition to the overall progress status. The status management table 100 and the examination device running status table 102 are displayed side by side, and the correspondence relationship information indicates the relationship of the information displayed in both the tables 100 and 102. Therefore, the staff can easily and quickly grasp both the overall progress information and the individual progress information, as compared to displaying the tables 100 and 102 individually in separate systems.

In the status management table 100 which is an example of the first table, a plurality of stages are arranged in a sequence of time, and the first controller 6 moves an icon 100B, which is an example of a display indicating the current stage of each diseased animal, according to the progress status of each subject. Therefore, it is easy to intuitively grasp the overall progress status.

The correspondence relationship information includes the thick frame 100E (corresponding to the first mark) visually emphasizing the target diseased animal (corresponding to the target subject) in the status management table 100 (corresponding to the first table), and the thick frame 102E (corresponding to the second mark) visually emphasizing the examination device assigned to the target diseased animal in the examination device running status table 102 (corresponding to the second table). Since the first mark is displayed in the status management table 100 and the second mark corresponding to the first mark is displayed in the examination device running status table 102, it is easy to grasp the correspondence relationship. Although the thick frame has been described as examples of the first mark and the second mark, the form of the mark is not limited to the thick frame, and may be a form of hatching or a form of a figure such as a rhombus. It may also be in the form of text such as “in examination”.

In the examination stage display section 100C of the status management table 100, the thick frame 100E (corresponding to the first mark) is attached to a name of the diseased animal which is an example of the subject identification information of the target diseased animal (corresponding to the target subject). In addition, in the running information display section 102A of the examination device running status table 102, the thick frame 102E (corresponding to the second mark) is attached to the name of the target diseased animal. As described above, by correlating the first mark and the second mark with the subject identification information of the target diseased animal, the correspondence relationship can be more easily grasped.

In the above example, the status management table 100 and the examination device running status table 102 are displayed on both the operation input monitor 38 and the management monitor 40, but may be displayed on at least one of the operation input monitor 38 or the management monitor 40.

The status management table 100 and the examination device running status table 102 may be displayed on other display devices (the waiting room monitor 32, the check-up monitor 33, the check-up monitor 34, the operation input monitor 38, and the examination management monitor 42) and the like, in addition to at least one of the operation input monitor 38 or the management monitor 40. In a case of displaying the contents on these display devices, both of the status management table 100 and the examination device running status table 102 may not be displayed, or only one of them may be displayed.

In a case of displaying the contents on a display device other than the operation input monitor 38 and the management monitor 40, it is preferable that contents to be displayed are controlled according to a display device on which the contents are displayed or a place where the display device is disposed.

Particularly, since the waiting room monitor 32 is watched by a person other than the staff of the animal hospital 1, in a case where the status management table 100 is displayed on the waiting room monitor 32, it is preferable that detailed examination contents or dispensing contents, and information on personal information of the owner or the diseased animal are not displayed. Therefore, information indicating the current stage of each diseased animal may be displayed on the waiting room monitor 32, and it is preferable that only information on a part of the status management table 100 is displayed. As described above, it is preferable that the information indicating the current stage of each diseased animal is displayed as a part of the status management table 100 on the waiting room monitor 32 in order for the owner who waits for in the waiting room 10 to check the current status. For example, since such display is performed, even in a case where the check-up or examination is crowded, the owner can recognize how much the check-up or examination is crowded or a rough estimate of the waiting time.

As described above, in a case where the contents are displayed on the display device other than the operation input monitor 38 and the management monitor 40, in a case where the contents to be displayed are controlled according to the display device on which the contents are displayed or the place in which the display device is disposed, a person stays in a range in which the display on the display device can be watched may be captured, face recognition processing may be performed on the captured image, it may be determined whether or not there is the staff of the animal hospital 1, and the contents to be displayed may be changed between a case where there is only the staff and a case where there is a person other than the staff such as the owner.

Although the display device independent of the operation terminal 24 has been described as an example of the display device, the display device may be a display device provided integrally with the operation terminal 24.

In the above example, a photo of the diseased animal, which is an example of subject identification information, is shown in icon 100B indicating the current stage of the diseased animal, but the name of the diseased animal or the like may be used in addition to the photo of the diseased animal. Note that, it is considered that there are many staff who remember the diseased animals in appearance. Therefore, for the icon 100B, it is more preferable to use a photo of the diseased animal than to use the name of the diseased animal.

Even in a case where the status management table 100 is displayed on the waiting room monitor 32, it is preferable to use the photo of the diseased animal for the icon 100B. This is because it is considered that the owner can easily check his or her own diseased animal in a case where the photo of the diseased animal is displayed.

In addition, information other than the status management table 100 and the examination device running status table 102 may be additionally displayed. For example, in a case where the status management table 100 is displayed on the check-up monitor 33, the results of the examinations using the examination device group 44 and the radiological examination device 45 may be displayed as information for describing the check-up to the owner. For example, in a case where a diseased animal is selected from the status management table 100, a window for displaying the result of the examination is opened. Examples of the results of the examination include the result of a blood examination, a radiographic image, and an ultrasound image.

Furthermore, information other than the result of the examination may be added. As such information, for example, information indicating the remaining amount of consumables in each examination device of the examination device group 44, the radiological examination device 45, and the like may be considered.

In addition, for example, in a case where the name 120 of the examination device is selected by the staff by using the operation input monitor 38 and the input unit 86 of the management monitor 40, the examination management terminal 26 may display various information on the selected examination device on the running information display section 102A. Examples of various information in this case include the remaining amount of consumables, information on an occurred abnormality in a case where the abnormality occurs in the examination device, or contact information such as a serviceman who performs maintenance on the examination device.

Although it has been described in the present embodiment that the IC tag is included in the check-up card, the IC tag may be provided separately from the check-up card. For example, the IC tag may be provided so as to be attachable on the diseased animal, specifically, so as to be attachable on a collar of the diseased animal.

A tag having the Global Positioning System (GPS) therein may be used instead of the IC tag. In this case, in a case where the reception is performed in the animal hospital 1, the tag having the GPS therein may be attached to the diseased animal, and the operation terminal 24 may perform control for displaying a stage (for example, the current stage is the stage of “check-up/treatment” in a case where the position of the diseased animal is the first check-up room 11) corresponding to the position of the diseased animal as the current stage based on positional information of the diseased animal transmitted from the tag having the GPS therein.

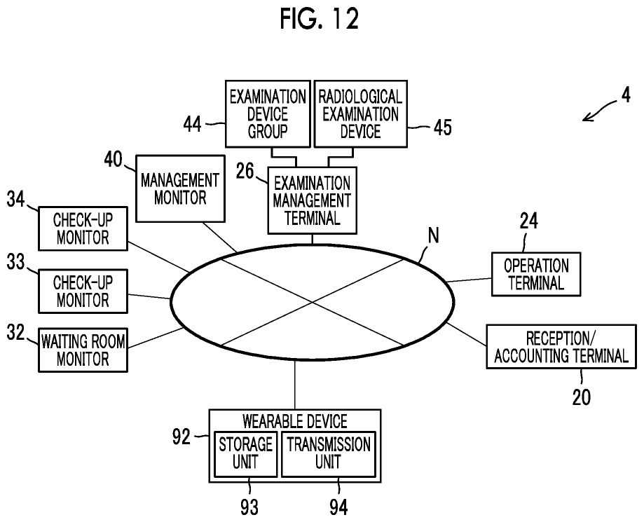

It has been described that the current stage of diseased animal is updated by using the IC tag and the tag readers 50, 51, 52, 54, 56, 58, and 59. However, instead of or in addition to this example, in a case where the operation terminal 24 receives predetermined information from a wearable device used by the staff or the owner of the diseased animal, the current stage of the diseased animal may be updated. An example of the configuration of the hospital support system 4 in this case is shown in FIG. 12. A wearable device 92 comprises a storage unit 93 that stores information described regarding the diseased animal, such as the description in the medical chart of the diseased animal, and a transmission unit 94 that transmits the information stored in the storage unit 93, and is communicably connected to the network N. As a specific example of the wearable device 92, there are a so-called electronic pen, a camera attachable to eyeglasses worn by the staff, and a camera integrated with eyeglasses.

The display modes of the status management table 100, which is an example of the first table, and the examination device running status table 102, which is an example of the second table, are not limited to the above example, and can be variously modified as shown in the following modification examples or embodiments. In the following modification examples and embodiments, the same reference signs will be assigned to the same configurations as those in the above example and descriptions thereof will be omitted, and differences will be mainly described.

Modification Example 1 of First Embodiment

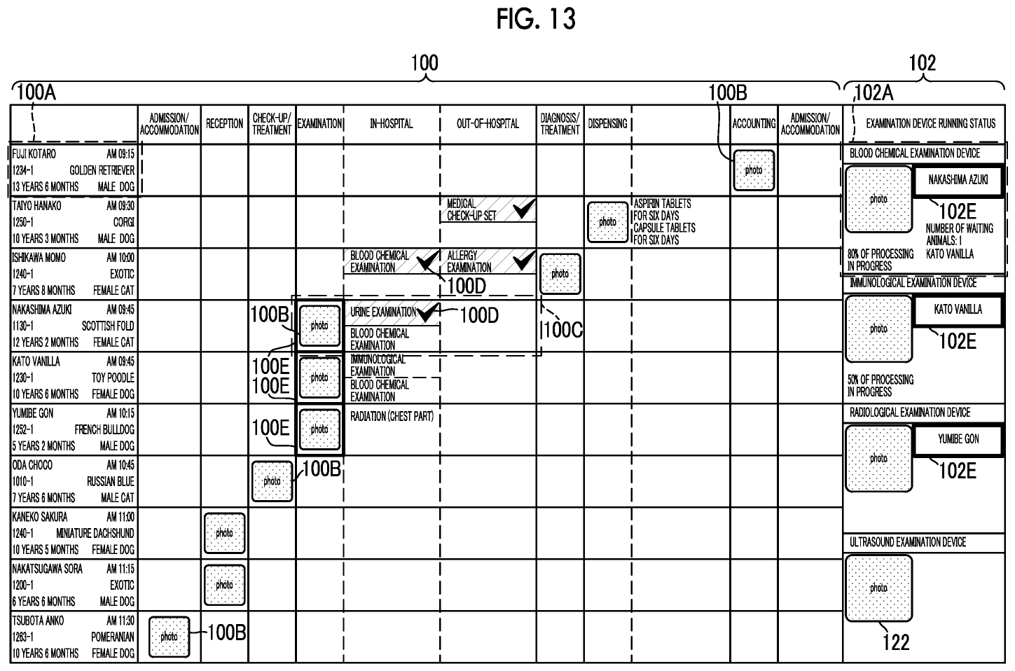

In Modification Example 1 of the first embodiment shown in FIG. 13, the target to which the first mark is attached in the examination stage display section 100C of the status management table 100 differs from the above-described example. That is, in the above-described example, the thick frame 100E is attached to the examination item in the examination stage display section 100C, but in Modification Example 1, the thick frame 100E is attached to the icon 100B. The other points are the same as the above-described example. As described above, the target to which the first mark is attached may not be an examination item, and may be subject identification information such as a photo of a target diseased animal. As in the above example, even in a case where the thick frame 100E is not attached to the examination item in the examination stage display section 100C, in a case where the thick frame 100E is attached to the icon 100B of the target diseased animal, by checking the thick frame 102E displayed in the examination device running status table 102, it is possible to grasp which examination is being performed.

Modification Example 2 of First Embodiment

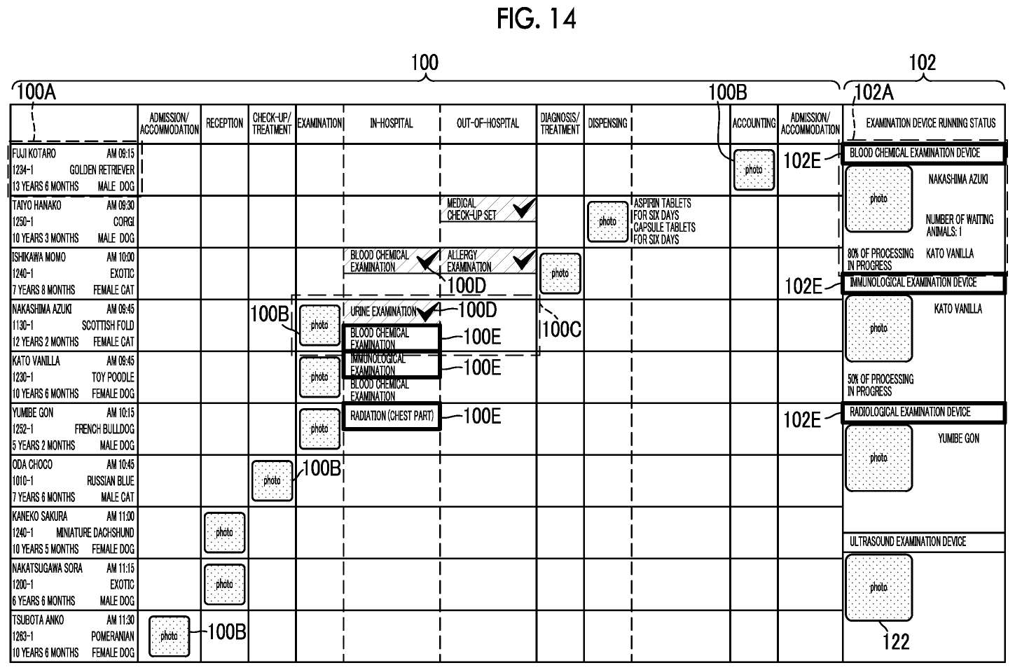

In Modification Example 2 of the first embodiment shown in FIG. 14, the target to which the second mark is attached in the running information display section 102A of the examination device running status table 102 differs from the above-described example. That is, although the thick frame 102E is attached to the name of diseased animal (an example of subject identification information) in the running information display section 102A in the above-described example, the thick frame 102E is attached to the name 120 of the examination device (see FIG. 10) in the running information display section 102A in Modification Example 2. The other points are the same as the above-described example. The name 120 of the examination device is an example of device identification information for identifying the device. As described above, the target to which the second mark is attached may be not the subject identification information in the running information display section 102A, and may be the device identification information. As the device identification information, the model number of the device may be used instead of the name.

### Second Embodiment

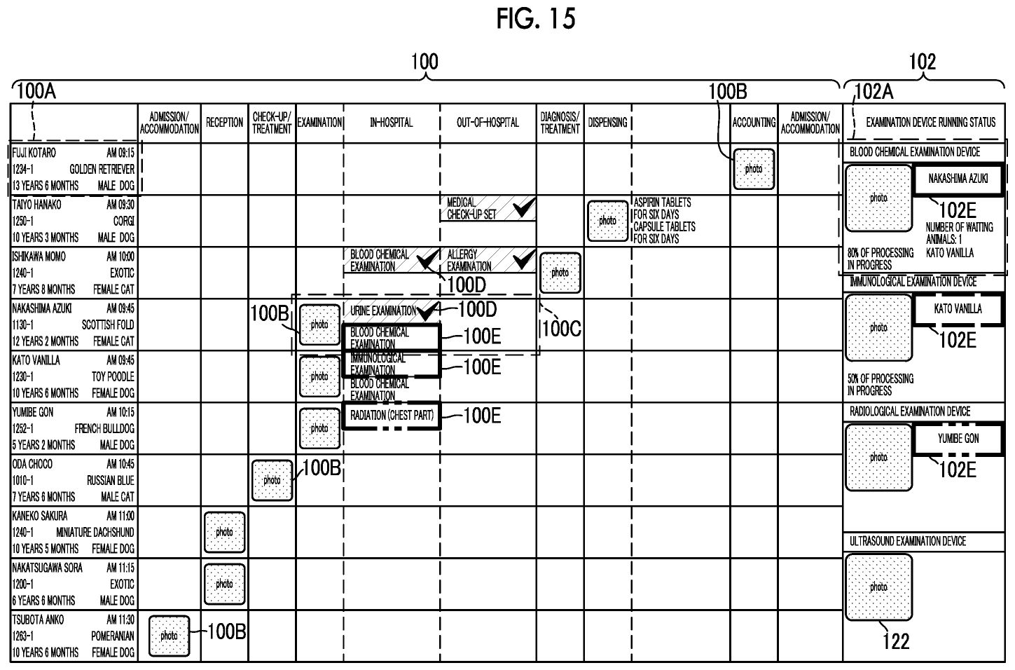

In the second embodiment shown in FIG. 15, the first mark and the second mark are displayed in display modes that can be identified for each target diseased animal. In the example shown in FIG. 15, the colors of the thick frame 100E, which is an example of the first mark, and the thick frame 102E, which is an example of the second mark, are changed for each target diseased animal. Specifically, the colors of the thick frame 100E and the thick frame 102E are different for each diseased animal of “Nakashima Azuki”, “Kato Vanilla”, and “Yumibe Gon”. In FIG. 15, the fact that the thick frame 100E and the thick frame 102E are displayed in different colors is indicated by changing the line type (a solid line, a dotted line, or a dashed-two dotted line).

As described above, by displaying in a display mode that can be identified for each target diseased animal (the target subject), the correspondence relationship of each target diseased animal becomes clearer. As an example of changing the display mode, in addition to the changing of the color, for example, a method of changing the line type, or changing the shape of the mark and the type of hatching which are attached to each display mode may be considered. Any of these methods may be used. In a case where the thick frame 100E and the thick frame 102E are blinked and displayed, the blinking interval may be changed for each target diseased animal.

### Third Embodiment

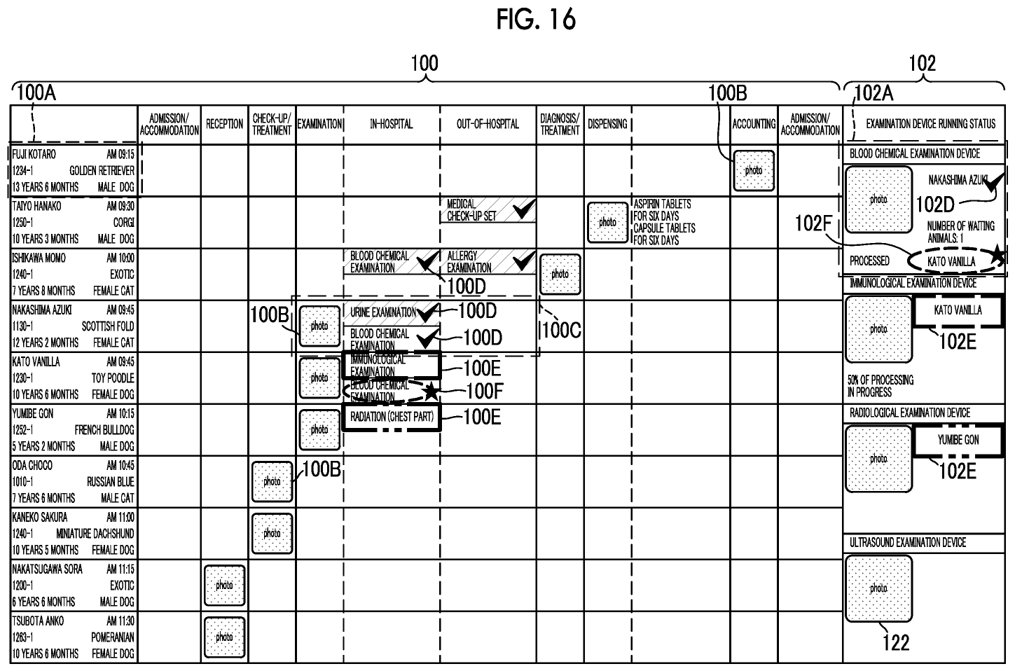

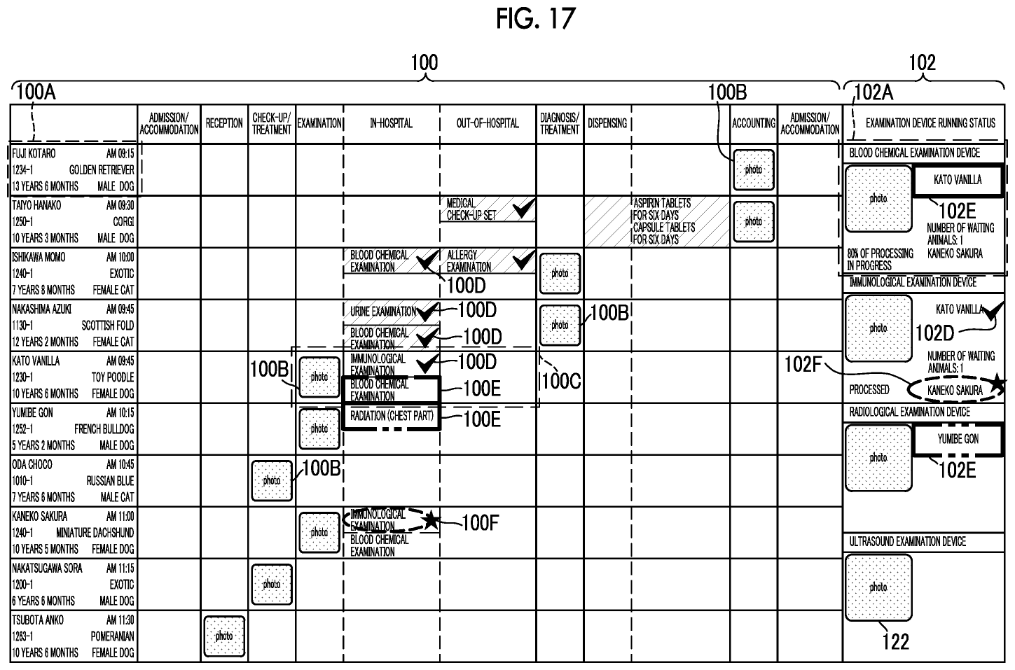

In the third embodiment shown in FIGS. 16 and 17 in addition to FIG. 15, the display mode of the second mark is changed according to the state indicating the progress of the examination in a running information display section 102A, and the display mode of the first mark is changed in conjunction with the second mark.

In FIG. 16, in the running information display section 102A of the blood chemical examination device, a check mark 102D is attached to “Nakashima Azuki” which is the name of the target diseased animal. The check mark 102D is an example of the second mark indicating a state in which the examination is completed. The check mark 102D corresponds to the check mark 100D of the examination stage display section 100C.

In addition, in the running information display section 102A of the blood chemical examination device, a star mark 102F composed of a dashed line frame of the oval shape and a star shape is attached to “Kato Vanilla” which is the name of the waiting target diseased animal. The star mark 102F is an example of the second mark indicating a state in which the examination is being prepared. The star mark 102F corresponds to the star mark 100F of the examination stage display section 100C. The star mark 100F is an example of the first mark indicating that the examination is being prepared.

The thick frame 100E and the thick frame 102E is examples of the first mark and the second mark indicating the state in examination as described above, respectively. Therefore, the three display modes of the thick frames 100E and 102E, the star marks 100F and 102F, and the check marks 100D and 102D can indicate the three states of the examination being performed, the examination being prepared, and the examination being completed.

As described above, in the third embodiment, in the running information display section 102A, the display mode of the second mark is changed according to the state of examination. Then, in conjunction with the change in the display mode of the second mark, the display mode of the first mark of the examination stage display section 100C is also changed. The CPU 60 of the operation terminal 24 obtains information on a state of the examination from the examination management terminal 26.

Examples of such processing are shown using FIGS. 15 to 17. First, FIG. 15 is set as the reference state. In FIG. 15, the blood chemical examination is being performed for “Nakashima Azuki”, the immunological examination is being performed for “Kato Vanilla”, and the radiological examination is being performed for “Yumibe Gon”. In this state, in each running status display section 102A, the thick frame 102E indicating a state in which the examination is being performed is attached as the second mark. In conjunction with the second mark, the thick frame 100E is attached to the examination item of the “blood chemical examination” also in the examination stage display section 100C of “Nakashima Azuki”. In addition, the thick frame 100E is attached to the examination item of the “immunological examination” in the examination stage display section 100C of “Kato Vanilla”. The thick frame 100E is attached to the examination item of the “radiological examination” in the examination stage display section 100C of “Yumibe Gon”.

In FIG. 16 showing the state after time passes from the state shown in FIG. 15, the blood chemical examination of “Nakashima Azuki” is completed. Therefore, in the running information display section 102A, the second mark attached to “Nakashima Azuki” is changed from the thick frame 102E to the check mark 102D indicating a state in which the examination is completed. Then, for the waiting “Kato Vanilla”, the star mark 102F indicating a state in which the examination is being prepared is attached. In conjunction with the change in the display mode of the second mark in the running information display section 102A, also in the examination stage display section 100C, the check mark 100D is attached to the examination item of the blood chemical examination of “Nakashima Azuki”, and the star mark 100F is attached to the examination item of the blood chemical examination of “Kato Vanilla”.

In FIG. 17 showing the state after time passes from the state shown in FIG. 16, the blood chemical examination of “Kato Vanilla” is started while the immunological examination of “Kato Vanilla” is completed. Therefore, in the running information display section 102A of the blood chemical examination, the second mark attached to “Kato Vanilla” is changed from the star mark 102F indicating a state in which the examination is being prepared to the thick frame 102E. In addition, in the running information display section 102A of the immunochemical examination, the check mark 102D indicating a state in which the examination is completed is attached to “Kato Vanilla”, and the star mark 102F indicating a state in which the examination is being prepared is attached to “Kaneko Sakura”. In conjunction with the change in the display mode of the second mark in the running information display section 102A, also in the examination stage display section 100C, the thick frame 100E is attached to the examination item of the blood chemical examination of “Kato Vanilla”, and the check mark 100D is attached to the examination item of the immunological examination of “Kato Vanilla”. In addition, the star mark 100F is attached to the examination item of the immunological examination of “Kaneko Sakura”.

As in the second embodiment, it is easy to grasp the progress status in the examination stage by changing the display mode of the second mark according to the state of examination and changing the first mark in conjunction with the change in the display mode of the second mark. As the state of the examination, in addition to a first state (indicated by the thick frames 100E and 102E) which is a state in which the examination is being performed, by indicating which of a second state (indicated by the star marks 100F and 102F) which is a state in which the examination is being prepared and a third state (indicated by the check marks 100D and 102D) which is a state in which the examination is completed, the state of the examination and the running information of the examination device can be grasped in more detail. By grasping such detailed information, for example, it is easy for the staff to change the schedule flexibly, such as the changing the order of examination items by grasping the congestion status of the examination device.

In addition, in the examination stage display section 100C of “Nakashima Azuki” in FIG. 16, although the check mark 100D indicating the state of completion of the examination is attached to the examination item of the blood chemical examination, it is not hatched unlike the urine examination in the upper row. This is because the name of “Nakashima Azuki” still remains in the running information display section 102A of the blood chemical examination, in a case where the “Nakashima Azuki” disappears from the running information display section 102A, the examination item of the blood chemical examination of the examination stage display section 100C is hatched in addition to the check mark 100D as in the case of the urine examination.

That is, the state in which hatching is not attached and only the check mark 100D is attached is an immediately-after-completion state, after the examination for a certain target diseased animal is completed and before the examination for the next target diseased animal is started in the examination device. As described above, the immediately-after-completion state (corresponding to a fourth state) may be included as the state of the examination. Accordingly, since the state of the examination can be displayed in more detail, it is easy to grasp the progress status of the examination in detail.

In FIG. 17, for “Nakashima Azuki” in which two examination items scheduled to be performed are all completed, since the stage of the examination is completed, the icon 100B is transferred to the stage of the diagnosis/treatment which is the next stage.

### Fourth Embodiment

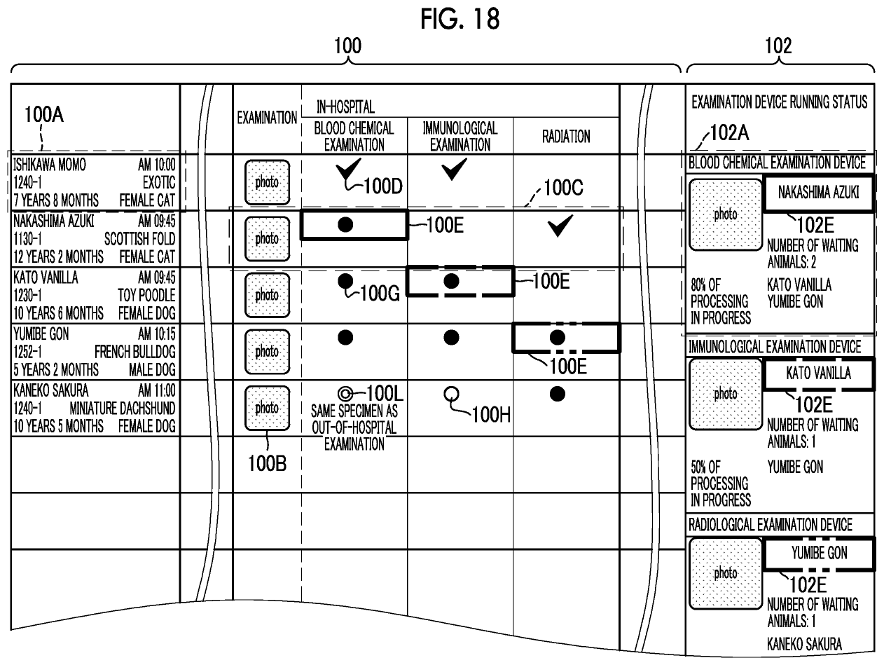

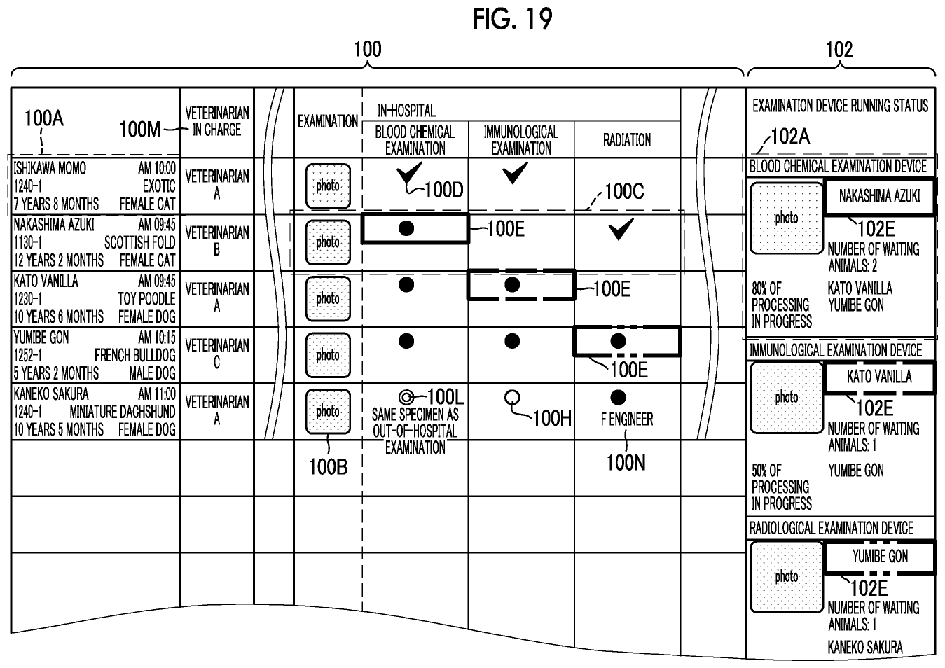

The fourth embodiment shown in FIGS. 18 and 19 is different in the form of the examination stage display section 100C. Specifically, in a case where there are a plurality of examination items scheduled to be performed for one target diseased animal, in the above embodiments, the plurality of examination items are arranged in the vertical direction orthogonal to the time axis, but in the fourth embodiment, the plurality of examination items are arranged in the horizontal direction parallel to the time axis.

In a case where there is a margin in the width of the display screen and the examination items are set in the horizontal direction as described above, the examination items can be arranged along the time axis, and thus the display may be easy to see.

In the example shown in FIG. 18, in the examination stage display section 100C, a black circle 100G, a white circle 100H, and a double circle 100L are attached in addition to the thick frame 100E indicating a state in examination or the check mark 100D indicating a completion state of the examination. In a case where the specimen collected from the living body is an examination target, a state such as waiting for collection of the specimen occurs. The black circle 100G indicates that the specimen has been collected, and the white circle 100H indicates the state of waiting for collection of the specimen. The double circle 100L indicates the state of waiting for collection of the specimen as in the case of the white circle 100H, and indicates a case of using the same specimen in another examination.

As shown in the example of FIG. 18, the white circle 100H and the double circle 100L indicate the state of waiting for collection of the specimen, thereby providing the following advantages. That is, “Kaneko Sakura” in the example shown in FIG. 18 is scheduled to be subjected to the radiological examination that does not require collection of the specimen, in addition to the blood chemical examination and the immunological examination waiting for collection of the specimen. In such a case, wasteful waiting time can be eliminated by performing the radiological examination prior to the blood chemical examination and the immunological examination. In addition, since the free time for the radiological examination is reduced, improvement in the running rate of the radiological examination device can also be expected.

As in the example shown in FIG. 18, the double circle 100L indicates the presence of another examination using the same specimen, so that it is possible to expect an effect that the number of specimen collection can be reduced by performing the specimen collection at one time.

In addition, as in the example shown in FIG. 19, it is possible to easily grasp a person in charge by adding a display 100M of the veterinarian in charge and/or a display 100N of the engineer in charge.

### Fifth Embodiment

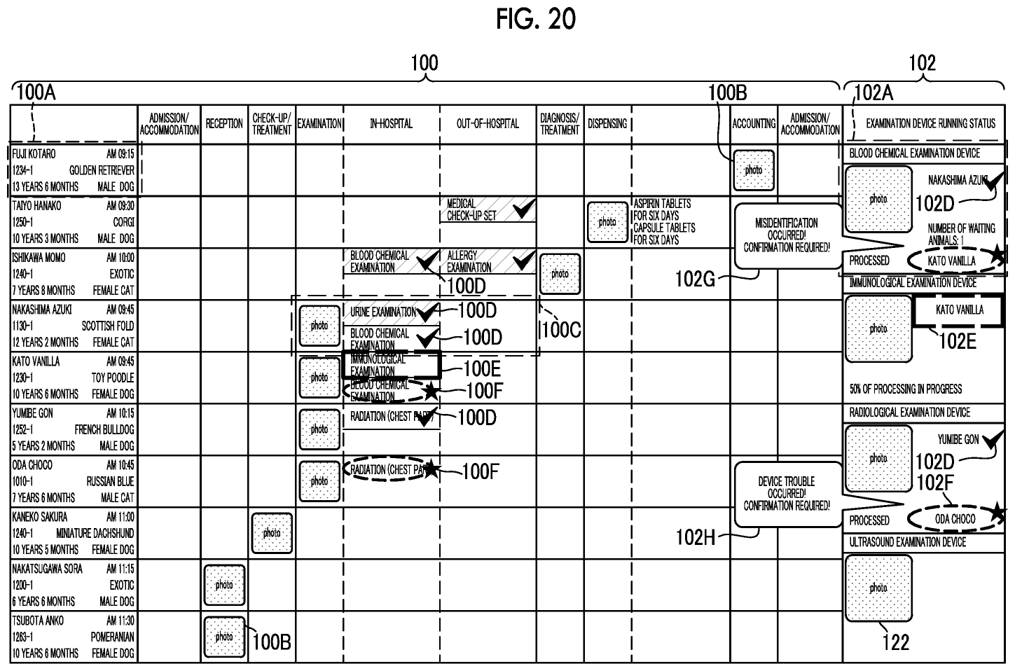

The fifth embodiment shown in FIG. 20 is an example showing error messages 102G and 102H indicating an error occurring for each examination device in the running information display section 102A. The error message 102H is an error message indicating an error that occurs in the examination device itself. The error message 102G is an error message indicating an input mistake of an examination target (so-called misidentification of a specimen) to be set in the examination device. By displaying such an error message, it is possible to easily check the error occurring in the examination device. The CPU 60 of the operation terminal 24 obtains information on such an error from the examination management terminal 26.

As an action of the staff in a case where the error message is displayed, for example, the subsequent examination is suspended. In a case where the staff can handle the error message and recovery can be performed, the examination will be resumed after the recovery. In a case where the examination is resumed, the display of the error message disappears. In addition, during the recovery operation, error messages may be made to blink in a conspicuous manner.

As an error detection method of detecting the error of the misidentification of the specimen in the examination management terminal 26, for example, the following method can be considered. A case will be considered in which a radio frequency identification (RFID) tag or a bar code in which identification information of a diseased animal is recorded is provided in a container in which a specimen is accommodated. In this case, there is a method of detecting an error by collating the identification information read by the RFID tag in the examination device with the identification information included in the appointment information registered in advance.

In addition, as a method of preventing the misidentification of the specimen, for example, a colored label with a color-coded for each diseased animal is attached to a container in which the specimen is accommodated, and the color of the label is matched with the color of the first mark and the second mark such as the thick frame 100E and the thick frame 102E, which are color-coded for each diseased animal. In this way, the person in charge of the examination can visually collate the colors of the first mark and the second mark in the status management table 100 and the examination device running status table 102 with the label color of the container. The collation of the color is effective because it is easier to detect a match or mismatch as compared to collation of texts.

### Sixth Embodiment

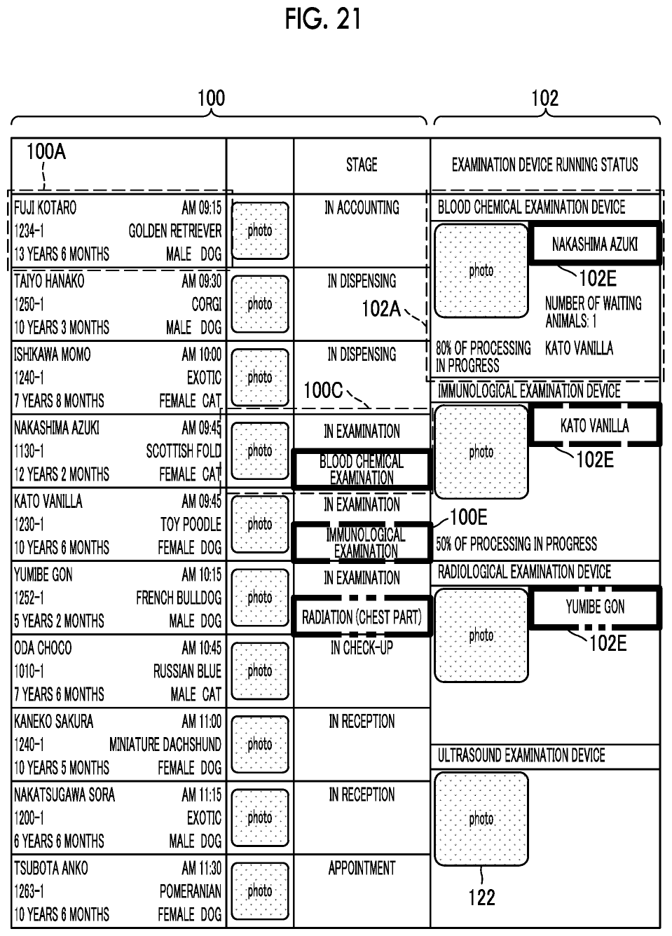

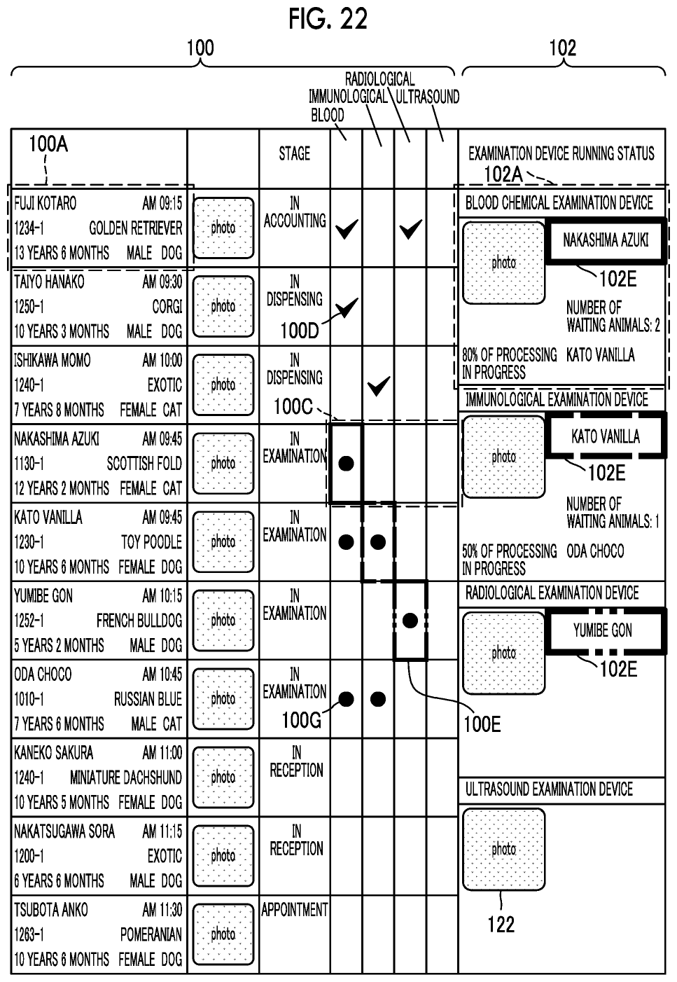

As in the sixth embodiment shown in FIGS. 21 and 22, in the status management table 100 which is an example of the first table, the plurality of stages may not be arranged in a sequence of time. In FIG. 21, each stage such as the reception stage, the examination stage, the dispensing stage, or the accounting stage is not arranged in a sequence of time, and only the display indicating the current stage is displayed in texts such as “in reception”, “in examination”, “in dispensing”, and “in accounting”. In a case where a plurality of stages are arranged in a sequence of time, the screen size becomes large. Therefore, in a case where the display is performed as shown in FIG. 21, it is possible to reduce the screen size. For this reason, it is suitable in a case where a portable terminal (a smart phone, a tablet terminal, or the like) with a small screen size is used as a display device. Such a portable terminal is, for example, a terminal carried by the staff in the hospital.

In addition, as shown in FIG. 22, in the examination stage display section 100C, the plurality of examination items may be arranged in a sequence of time. Other points are the same as FIG. 21. As described above, in a case where the examination items are arranged for each examination item, it is convenient because the running status of each examination item can be grasped. For example, the screen of the first embodiment shown in FIG. 8 and the screen of the sixth embodiment shown in FIG. 21 or 22 may be switched and used for each display device. In this way, for example, the display device with a relatively large screen can be used to display the screen of the first embodiment, and the portable terminal with a relatively small screen can be used to display the screen of the sixth embodiment.

In the above embodiments, the examination device running status table 102 is described as an example of the second table, and a medical treatment device may be used for the medical treatment other than the examination. In this case, as the second table, a medical treatment device running status table is provided instead of or in addition to the examination device running status table 102. Then, in the status management table 100, a medical treatment stage display section is provided instead of or in addition to the examination stage display section 100C. It is preferable to indicate the correspondence relationship of both the first mark and the second mark by displaying the first mark and the second mark to the medical treatment stage display section.

Although it has been described in the present embodiment that the animal hospital 1 in which the medical treatment is performed on the animal other than the person is used as an example of the hospital to which the hospital support system 4 is applied and the animal (diseased animal) other than the person is used as the subject, the present invention is not limited to the present embodiment. The hospital in which the medical treatment is performed on the person may be used as the example of the hospital to which the hospital support system 4 is applied, and the person may be used as the subject.

The hardware configuration of the computer constituting the hospital support apparatus shown as the operation terminal 24 can be variously modified. For example, it is also possible to constitute the hospital support apparatus with a plurality of computers separated as hardware for the purpose of improving processing capacity and reliability. The functions of the first controller 6, the second controller 7, and the third controller 8 are distributed to and carried by three computers. In this case, the hospital support apparatus is constituted by three computers.

As described above, the hardware configuration of the computer can be appropriately changed in accordance with the required performance such as processing capacity, safety, and reliability. Furthermore, not only the hardware but also the hospital support program 63 which is an example of the operation program may be duplicated or be distributed to and stored in a plurality of storage devices (storage units) for the purpose of securing safety and reliability.

In the above embodiments, for example, as a hardware structure of a processing unit that executes various processes such as the first controller 6, the second controller 7, and the third controller 8, various processors described below can be used. As described above, in addition to the CPU which is a general-purpose processor that executes software (operation program) and that functions as various processing units, examples of the various processors include a programmable logic device (PLD) which is a processor capable of changing a circuit configuration after a field programmable gate array (FPGA) is manufactured and a dedicated electric circuit which is a processor having a circuit configuration designed as a dedicated circuit in order to perform specific processing such as an application specific integrated circuit (ASIC).

One processing unit may be constituted by one of various processors, or may be constituted by a combination of two or more processors of the same type or different types (for example, a combination of a plurality of FPGAs, and a combination of a CPU and an FPGA). In addition, a plurality of processing units may be constituted by one processor.

As an example in which the plurality of processing units are constituted by one processor, as represented by a system on chip (SoC) or the like, there is a form of using a processor that realizes functions of the entire system including a plurality of processing units with one integrated circuit (IC) chip. Thus, various processing units are constituted by using one or more of the above-described various processors as a hardware structure.

More specifically, as a hardware structure of these various processors, an electrical circuit (circuitry) in which circuit elements such as semiconductor elements are combined can be used.

From the above description, it is possible to grasp the hospital support apparatus in the following additional item.

Additional Item

A hospital support apparatus comprising:

a first control processor that performs control for displaying a first table on a display device, the first table displaying a current stage of a subject among a plurality of stages that the subject goes through in a hospital from arrival of the subject at the hospital to completion of accounting for each of the subjects, the stage including at least one of medical treatment or examination;

a second control processor that performs control for displaying a second table side by side with the first table on the display device, the second table displaying running information indicating a running status of each of a plurality of devices used for the medical treatment or the examination for each device; and

a third control processor that performs control such that the first table and the second table display correspondence relationship information indicating a correspondence relationship between a target subject of which the current stage indicated in the first table is a stage of the medical treatment or the examination, and the device assigned to the target subject in the second table.

Although it has been described in the above embodiment that the hospital support program 63 is stored (installed) in the storage unit 62 in advance, the examination management program 73 is stored in the storage unit 72 in advance, and the display control program 83 is stored in the storage unit 82 in advance, the present invention is not limited thereto. Each of the hospital support program 63, the examination management program 73, and the display control program 83 may be stored in a recording medium such as a compact disk read only memory (CD-ROM), a digital versatile disk read only memory (DVD-ROM), and a universal serial bus (USB) memory. Each of the hospital support program 63, the examination management program 73, and the display control program 83 may be downloaded from an external device through a network.

What is claimed is:

1. A hospital support apparatus comprising:
a first controller that performs control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination;
a second controller that performs control for displaying a second table side by side with the first table on the display device, the second table displaying running information indicating a running status of each of a plurality of devices used for the medical treatment or the examination for each device; and
a third controller that performs control such that the first table and the second table display correspondence relationship information indicating a correspondence relationship between a target subject of which the current stage indicated in the first table is a stage of the medical treatment or the examination, and the device assigned to the target subject in the second table.

2. The hospital support apparatus according to claim 1,
wherein in the first table, the plurality of stages are arranged in a sequence of time, and
the first controller moves a display indicating the current stage of each subject according to a progress status of each subject.

3. The hospital support apparatus according to claim 1,
wherein the correspondence relationship information includes a first mark visually emphasizing the target subject in the first table, and a second mark visually emphasizing the device assigned to the target subject in the second table.

4. The hospital support apparatus according to claim 3,
wherein, regarding the target subject, the first table displays at least one of subject identification information of the target subject or an examination item scheduled to be performed for the target subject, and
the first mark is attached to at least one of the subject identification information or the examination item.

5. The hospital support apparatus according to claim 3,
wherein the second table displays subject identification information of the subject assigned to each device in addition to device identification information of the device, and
the second mark is attached to at least one of the device identification information or the subject identification information.

6. The hospital support apparatus according to claim 3,
wherein the first mark and the second mark are displayed in a display mode in which each of the target subjects is identified.

7. The hospital support apparatus according to claim 3,
wherein, by changing a display mode of the second mark for each of a plurality of the target subjects assigned to the device, the second table indicates a state of each target subject among a plurality of the states including a first state in which the medical treatment or the examination corresponding to the device is being performed, a second state in which the medical treatment or the examination corresponding to the device is being prepared, and a third state in which the examination is completed, and
a display mode of the first mark is also changed in conjunction with the display mode of the second mark.

8. The hospital support apparatus according to claim 7,
wherein the plurality of states include a fourth state, and
the fourth state is an immediately-after-completion state, after the medical treatment or the examination of the target subject is completed and before a medical treatment or an examination of a next target subject is started in the device.

9. The hospital support apparatus according to claim 7,
wherein the first table displays a plurality of examination items in a display section of the stage of the examination.

10. The hospital support apparatus according to claim 9,
wherein in the display section, the plurality of states are displayed for each of the plurality of examination items.

11. The hospital support apparatus according to claim 1,
wherein the second table displays an error message indicating an error occurring for each device.

12. The hospital support apparatus according to claim 11,
wherein the error message includes at least one of an error message indicating an error occurring in the device or an error message indicating an input mistake of an examination target to be set in the device.

13. A non-transitory computer-readable storage medium storing an operation program of a hospital support apparatus causing a computer to function as the hospital support apparatus comprising:
a first controller that performs control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination;
a second controller that performs control for displaying a second table side by side with the first table on the display device, the second table displaying running information indicating a running status of each of a plurality of devices used for the medical treatment or the examination for each device; and
a third controller that performs control such that the first table and the second table display correspondence relationship information indicating a correspondence relationship between a target subject of which the current stage indicated in the first table is a stage of the medical treatment or the examination, and the device assigned to the target subject in the second table.

14. An operation method of a hospital support apparatus, the method comprising:
a first control step of performing control for displaying a first table on a display device, the first table displaying a current stage of a subject among a plurality of stages that the subject goes through in a hospital from arrival of the subject at the hospital to completion of accounting for each of the subjects, the stage including at least one of a medical treatment or an examination;
a second control step of performing control for displaying a second table side by side with the first table on the display device, the second table displaying running information indicating a running status of each of a plurality of devices used for the medical treatment or the examination for each device; and
a third control step of performing control such that the first table and the second table display correspondence relationship information indicating a correspondence relationship between a target subject of which the current stage indicated in the first table is a stage of the medical treatment or the examination, and the device assigned to the target subject in the second table.
